(19) United States
(12) Patent Application Publication
(10) Pub. No.: US 2007/0161891 A1
(43) Pub. Date: Jul. 12, 2007

(54) In vivo brain elasticity measurement by magnetic resonance elastography with vibrator coil

(75) Inventors: David F. Moore, Rockville, MD (US); Seth Goldstein, Bethesda, MD (US); Randall Pursley, Germantown, MD (US); Lalith Talagala, Rockville, MD (US)

Correspondence Address: TOWNSEND AND TOWNSEND AND CREW, LLP, TWO EMBARCADERO CENTER, 8TH FLOOR, SAN FRANCISCO, CA, 94111, US

(73) Assignee: The Government of the United States of America, as represented by The Secretary of Health and Human, Rockville, MD (US)

(21) Appl. No.: 10/550,757

(22) PCT Filed: Mar. 25, 2004
(86) PCT No.: PCT/US04/09426

§ 371 Date: Oct. 19, 2006

Related U.S. Application Data
(60) Provisional application No. 60/458,681, filed on Mar. 27, 2003.

(51) Int. Cl. A61B 5/05

(52) U.S. Cl. 600/421

(57) ABSTRACT

A vibrator coil (10) is applied to the skull (30) by adaptation of a commercially available transcranial Doppler monitoring harness (40) during MR applies mechanical waves in the acoustic waves through the skull to the brain. Utilizing magnetic resonance elastography (MRE), non-invasive estimation of tissue elastic properties in three dimensions occurs. The propagation of the acoustic waves through brain tissue, coupled to phase alteration of voxel isochromats in the presence of applies motion encoding magnetic field gradients allows measurements of brain elasticity.

### CROSS-REFERENCES TO RELATED APPLICATIONS

This application claims priority from U.S. Provisional Patent Application Ser. No. 60/458,681 filed Mar. 27, 2003 entitled IN VIVO BRAIN ELASTICITY MEASUREMENT BY MAGNETIC RESONANCE ELASTOGRAPHY WITH VIBRATOR COIL by the inventors herein.

### STATEMENT AS TO RIGHTS TO INVENTIONS MADE UNDER FEDERALLY SPONSORED RESEARCH AND DEVELOPMENT

Not Applicable

### REFERENCE TO A “SEQUENCE LISTING,” A TABLE, OR A COMPUTER PROGRAM LISTING APPENDIX SUBMITTED ON A COMPACT DISK

Not Applicable

This invention relates to magnetic resonance elastography (MRE). Specifically, a vibrator coil imparts vibration to the brain during MR. The imparted vibrations allow a non-invasive determination of brain tissue elasticity for diagnosis of patient risk for malignant brain edema and herniation following acute brain trauma, stroke, intra-cerebral hemorrhage and other brain disease.

### BACKGROUND OF THE INVENTION

The mechanical properties of biological tissue often change during pathological processes. This is probably nowhere more evident that in neurological disorders, where as a consequence of brain encasement in the rigid skull vault, any brain tissue swelling may have little room for compensation. Disturbances of brain tissue elastance or compliance by trauma, stroke, infection or neoplasm results in alteration of the intra-cranial pressure and pressure-volume curve. (Thompson W. Intracranial Hypertension. In: Oh T, ed. Intensive Care Manual. London: Butterworths, 1990.) Compensatory treatment may result in a decrease in the intra-cranial blood volume, a decrease in CSF volume or osmotic shrinkage of more elastic brain tissue using mannitol or hypertonic saline osmotic pressure gradients.

Acute stroke affects about 500,000 people a year (Wolf P, D'Agostino, R B. Epidemiology of Stroke. In: Barnett H, Mohr, J P, Stein, B M, Yatsu, F M., ed. Stroke. New York: Churchill Livingstone, 1998:3-28.) and is the third most common cause of death in the United States. About 10% of all strokes involve occlusion of the middle cerebral artery with hemispheric infarction (Bogousslavsky J, Van Melle, G, Regli, E. The Lussanne Stroke Registry: Analysis of 1000 consecutive patients with first stroke. Stroke 1988; 19:1083-1085; Sacco R, Toni, D, Mohr, J P. Classification of Ischemic Stroke. In: Barnett H, Mohr, J P, Stein, B M, Yatsu, F M., ed. Stroke. New York: Churchill Livingstone, 1998:341-400.). About 10% of these patients develop coma and malignant brain edema (MBE) (Melo T, de Mendonca, A, Crespo, M, Carvalho, M, Ferro, J M. An emergency room-based study of stroke coma. Cerebrovascular Diseases 1992; 2:93-101); with an associated mortality of ˜80% (Bushnell C, Phillip-Bute, B G, Laskowitz, D T, et al. Survival and outcome after endotracheal intubation for acute stroke, Neurology 1999; 52:1374-1380.). MBE is secondary to tissue swelling due to increased cell water content following ischemia and cellular metabolic failure. Such swelling may results in herniation of brain tissue by a sub-falcine, transtentorial, tonsillar or rostral-caudal mechanism causing compression and compound damage to non-ischemic brain tissue. In the case of ischemic damage, maximal brain swelling usually occurs within 3-5 days post onset of stroke. Another cause of stroke related abnormal brain swelling and edema are intra-cerebral bleeds.

Head injury and associated brain trauma are also major public health problems being a major cause of mortality and morbidity in the 1-44 year age group (Kraus J, McArthur, D L, Silverman, T A, Jayaraman, M. Epidemiology of Brain Injury. In: Narayan R, Wilberger, J E, Povlishock, J T., ed. Neurotrauma. New York: McGraw-Hill, 1996.). After head trauma brain swelling may result in unequal brain compartment pressure gradients with resultant tissue shifts and herniation. Clinically the patient often demonstrates a deteriorating level of consciousness together with more localizing neurological signs. The timing of best medical therapy and surgical decompression in brain herniation syndromes is unclear. In some categories of stroke such as ischemic cerebellar ischemic stroke or hematoma surgical decompression has clearly been shown to be advantageous (Heros R. Cerebellar hemorrhage and infarction, Stroke 1982; 13:106-109; Jauss M, Krieger, D, Horning, C, Schramm, J, Busse, O. Surgical and medical management of patients with massive cerebellar infarctions: results of the German-Austrian Cerebellar Infarction Study, Journal of Neurology 1999; 246:257-264.). Other more investigative decompressive techniques such as hemicraniectomy and duroplasty have been shown in uncontrolled patient series to reduce patient mortality and morbidity following acute hemispheric stroke (Deleshaw J, Broaddus, W C, Kassell, N F, Haley, E C, Pendleton, G A, Vollmer, D G, Maggio, W W, Grady, M S. Treatment of right hemispheric cerebral infarction by hemicraniectomy. Stroke 1990; 21:874-881; Carter B, Oglivy, C S, Candia, G J, Rosas, H D, Buonanno, F; One-year outcome after decompressive surgery for massive nondominant hemispheric infarction. Neurosurgery 1997; 40:1168-1176.).

The optimal timing of intervention either medical or surgical is often unclear with clinical indices often having a low sensitivity and specificity in predicting timing of intervention (Schwab S, Steiner, T, Aschoff, A, Schwarz, S. Steiner, H H, Jansen, O, Hacke, W. Early hemicraniectomy in patients with complete middle cerebral artery infarction. Stroke 1998; 29:1888-1893).

MRE is a relatively recently implemented MR technique enabling non-invasive measurement of tissue elasticity by imaging alteration of the magnetic spin density caused by mechanical vibration or displacement wave propagation through deeper tissue with amplitudes in the order of a few micrometers. The technique is developing but has previously been used to examine breast and muscle tissue. By defining tissue elasticity MRE may provide unique imaging information of acute brain herniation syndromes allowing the design of more applied clinical studies where these questions could be systematically studied (Muthupillai R, Lomas, D J, Rossman, P J, Greenleaf, J F, Manduca, A, Ehman, R L. Magnetic resonance elastography by direct visualization of propagating acoustic strain waves, Science 1995; 269:1854-1857; Muthupillai R, Rossman, P J, Lomas, D J, Greenleaf, J F, Riederer, S J, Ehman, R L. Magnetic resonance imaging of transverse acoustic strain waves. Magnetic Resonance in Medicine 1996; 36:266-274; Van Houten E, Miga, M I, Weaver, J B, Kennedy, F E, Paulsen, K D. Three-Dimensional Subzone-Based Reconstruction Algorithm for MR Elastography; Magnetic Resonance in Medicine 2001; 45:827-837; Weaver J, Van Houten, EEW, Miga, M I, Kennedy, F E, Paulsen, K D. Magnetic resonance elastography using 3D gradient echo measurements of steady-state motion. Med. Phys. 2001; 28:1620-1628; Van Houten E, Paulsen, K D, Miga, M I, Kennedy, F E, Weaver, J B. An overlapping subzone technique for MR-based elastic property reconstruction. Magnetic Resonance in Medicine 1999; 42:779-786).

### BRIEF SUMMARY OF THE INVENTION

A vibrator coil is applied to the skull by adaptation of a commercially available transcranial Doppler monitoring harness during MR applies mechanical waves, in the auditory acoustic range, through the skull to the brain, typically at the temporal acoustic window of the skull. Utilizing magnetic resonance elastography (MRE), non-invasive estimation of tissue elastic properties in three dimensions occurs. The propagation of the acoustic waves through brain tissue results in transverse phase alteration of voxel isochromats allowing measurement of brain elasticity in the presence of applied magnetic field gradients. A protocol of timing for the acoustical excitation of the brain in a range from 125 hertz to 500 hertz is disclosed which includes synchronizing the acoustical interrogation to the subject's heart beat with a period of pre-excitation of the brain before the gating the interrogating radio frequency to the head of the patient. Image processing of the final data received from the MR scan includes image accumulation of phases in opposing directions, subtraction of the accumulated images to obtain a phase map, unwrapping of the phase map, to extract absolute phase and finally relating the phase to the original displacement to obtain the elastic properties of the brain being examined.

The clinical motivation for such measurements is to determine normal brain tissue compliance and the pathological alteration of brain tissue compliance or elasticity. Brain tissue compliance or elasticity alterations occur in neurological conditions such as brain trauma, acute stroke associated with malignant cytotoxic edema and in brain tumors associated with vasogenic edema. Tissue swelling results in altered mechanical properties while continued tissue swelling progressing to brain herniation often results in a reduced patient functional outcome. The present procedure leads to the non-invasive measurement of both the normal and altered brain compliance enabling the norm to be identified, compared to the abnormal, and allow identification and timely intervention for some of the above neurological conditions.

### BRIEF DESCRIPTION OF THE DRAWINGS

FIG. 1 is a perspective view of a patient showing the mounting bracket for the vibration inducing coil attached to the commercially available transcranial Doppler mounting harness. For clarity the coil is not shown (see FIG. 2);

FIG. 2 is an enlarged perspective view oriented the same as FIG. 1 of the vibration inducing coil held in the mounting bracket;

FIG. 3A is a view looking along the coil shaft, from the back of the head towards the front, showing the probe in contact with the acoustic window of a patient's head;

FIG. 3B is a view oriented the same as FIG. 3A, showing the mounting bracket holding the coil, illustrating the vibration inducing coil position for imparting acoustical vibration to the probe;

FIG. 3C is a view looking towards the skull along the coil axis showing the adjustable preload bar and torsion spring for applying adjustable torque to preload the probe against the acoustic window of the patients head;

FIG. 3D is a side elevation of FIG. 3C; FIG. 3 D is view looking from the top of the skull towards the bottom showing the mounting bracket and its attachment to the harness.

FIG. 4 schematically illustrates the synchronous trigger pulses, the motion-sensitizing gradient, the interrogating RF pulse, and the three-dimensional acquisition of data here schematically illustrating data acquisition of front to back sections taken through the skull;

FIG. 5 is a block diagram of the required processing for signals received from the MR device for determining the magnetic resonance elastography (MRE) of the brain being examined;

FIG. 6 is a block diagram of the sequence of triggering the coil with respect to the MRI machine;

FIG. 7 illustrates a patient at the head with a required “birdcage” about the head illustrating the collar with the vibration inducing coil mounted to his head within the volume defined by the bird cage, this illustration illustrating in the background the MR tunnel with the vibrator coil of FIG. 3A-3C hidden from view; and,

FIG. 8A to 8D illustrate a MRE single slice technique on a healthy volunteer showing sagittal slice acquisition with

FIG. 8A illustrating excitation frequency 125 Hz;

FIG. 8B illustrating Hilbert Transform;

FIG. 8C illustrating phase unwrapping of the Hilbert Transform; and,

FIG. 8D illustrating a Shear modulus map.

### DETAILED DESCRIPTION OF THE INVENTION

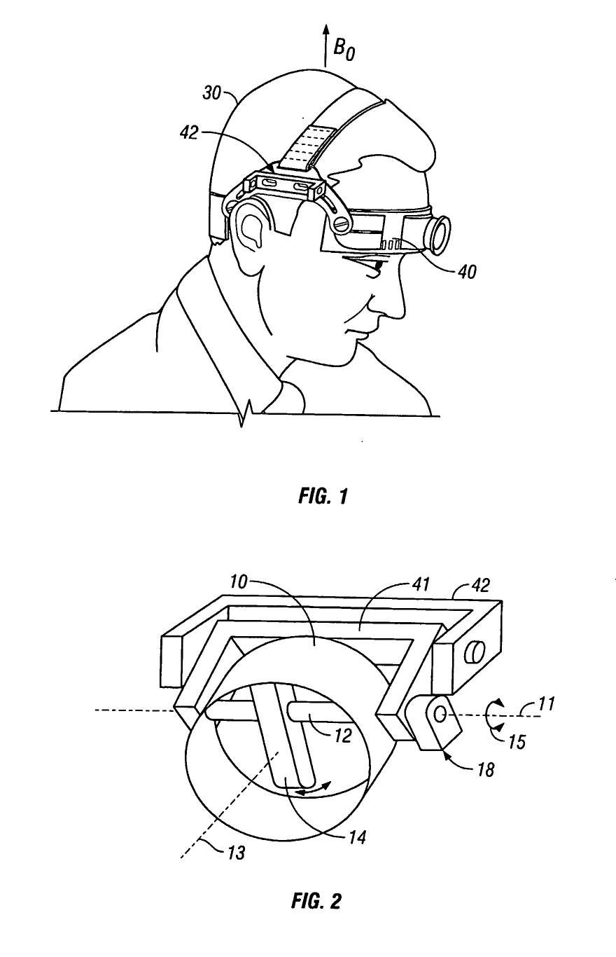

Referring to FIGS. 1 and 2, a small custom-built MR compatible coil 10 is placed about 5 cm from the temporal window so that torque, perpendicular to the static MR B₀ static field occurs after passing a small alternating current through the coil 10. The coil uses the B₀ static MRI magnetic field and the applied current to cause vibration of a contact probe 14 against the temporal window 31 of skull 30. This results in the generation of displacement waves within the skull and intra-cranial cavity causing displacement of tissue isochromats. Vibration frequencies in the range of 125-1000 Hz are used. The coil is comfortably applied to the skull 30 by adaptation of a commercially available transcranial Doppler monitoring harness 40 to hold a coil mounting bracket 42. Because of the presence of the ambient magnetic field B₀, coil 10 actuates without its own self contained magnet.

The standing wave field of mechanical stress required for MRI Elastography is produced by the vibrations of the small lightweight coil 10 mounted to an available ultrasonic transcranial Doppler device made MR compatible into holder 40 that attaches to the head as shown in FIG. 1 and FIG. 2. The coil is rigidly attached to a shaft 12 held in the coil support piece 41 which in turn is attached to bracket 42. The centerline 13 of the coil is oriented at approximately right angles to the B₀ static magnetic field, and when an alternating current from a remote, computer controlled power amplifier passes through the coil 10, it oscillates about an axis 11 coincident with the coil shaft 12 as indicated by the double arrow 15 in FIG. 2.

A contact probe 14 rigidly attached to shaft 12 touches the skull at the acoustic window 31 just in front of the upper ear. The coil 10 is designed so that a sinusoidal coil current at, for example, 250 Hz, results in a 250 Hz vibration applied to the skull. A torsion spring 16 wrapped around coil shaft 12 preloads the contact probe 14 against the skin. This preload is adjustable and operator defined by positioning and locking preload bar 18 so as to wind up torsion spring 16 to ensure optimal skull apposition force of the contact probe 14. An accelerometer (not shown) attached to the contact probe allows the vibration amplitude to be monitored. Lockable adjustments between parts 41 and 42 and the angle of the contact probe 14 on the coil shaft 12 are available to insure that the contact probe 14 is able to touch the skull at the acoustic window for different sized people while still maintaining the coil in the proper orientation with respect to B₀.

The coil 10 consists of 80 turns of ¼ mm diameter copper wire wound in a single layer onto a 3 mm thick, 5 cm diameter by 2.5 cm long plastic cylinder. An excitation current of 2 amperes at a frequency of 500 Hz (most demanding case) is predicted to produce an angular oscillatory amplitude of 4.5 milliradians. The corresponding motion at the end of the 3 cm long contact probe is approximately 0.1 mm when it barely touches the skin, and will be somewhat less when a preload is applied. Since this motion is applied at approximately 45 degrees to the surface, both compressive and shear stresses are applied to the skin. The accelerometer output divided by the excitation frequency provides a signal proportional to velocity and can be used to determine how to vary the current at different frequencies.

In practice, the coil support piece 41 will be adjusted and tightened in the mounting bracket 42 so that the contact probe 14 lines up with the acoustic window 31 in the anterior posterior direction. Next the head frame 40 will be adjusted and tightened. With the coil aligned perpendicular to B₀, contact probe 14 is rotated about the coil shaft 12 until it touches the skin at the acoustic window 31 and it is tightened on the shaft. (This may require adjustment of the coil support together with further tightening.) Finally the preload will be adjusted to the desired value by rotating and locking the preload bar 18 on the coil shaft so that the spring 16 pushes the contact probe 14 against the skull the desired amount.

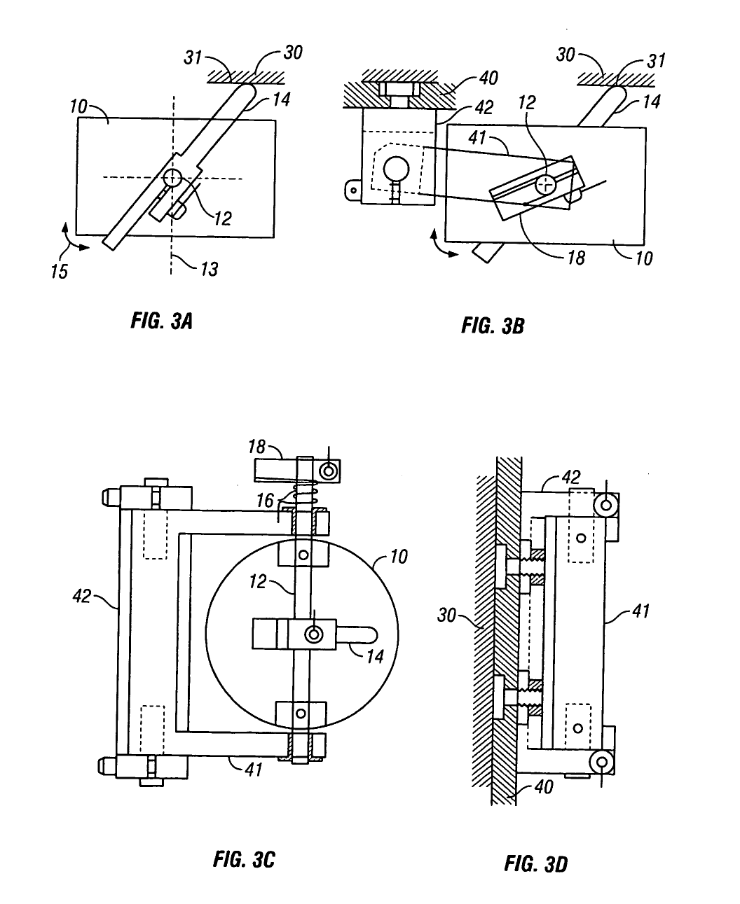

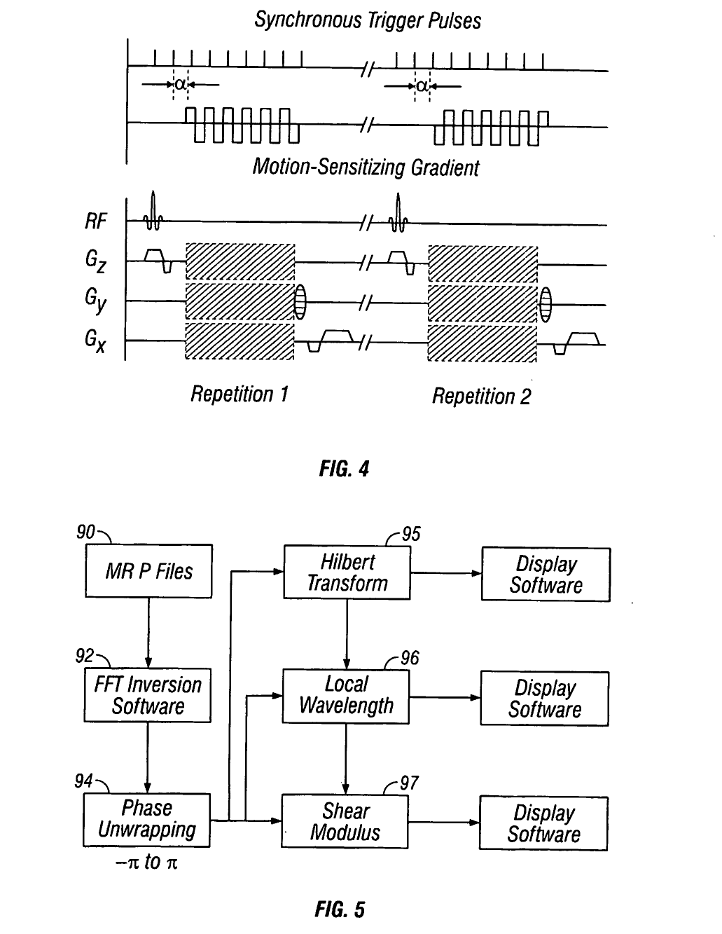

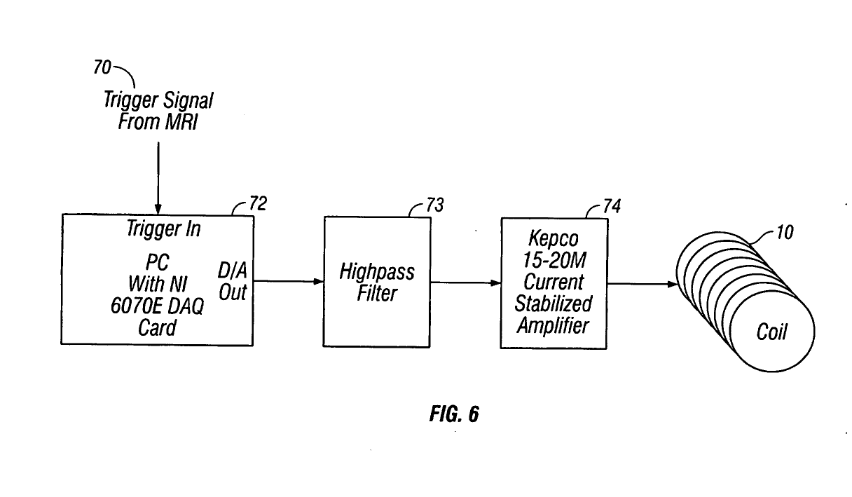

Previous work (by Muthupillai R, Lomas, D J, Rossman, P J, Greenleaf, J F, Manduca, A, Ehman, R L. Magnetic resonance elastography by direct visualization of propagating acoustic strain waves. Science 1995; 269:1854-1857 and Muthupillai R, Rossman, P J, Lomas, D J, Greenleaf, J F, Riederer, S J, Ehman, R L. Magnetic resonance imaging of transverse acoustic strain waves. Magnetic Resonance in Medicine 1996; 36:266-274) has demonstrated the principle of MRE in agarose phantoms and in human muscle. We adopt these techniques allowing direct visualization of acoustic strain waves by synchronized MR imaging with motion sensitizing gradients. Development of the NMR signal acquisition code takes place on a 1.5T Signa GE machine. A standard gradient echo sequence will be modified so that synchronized vibration-coil and gradient echo images are obtained (See FIG. 3 of the referenced article).

By using transmissible acoustic waves through the cranial cavity MR phase changes are obtained. Since they are sound waves in the range of 125-1000 Hz, they have no adverse biological consequences. A point from a sinusoidal signal generator will be used to trigger coil vibrations to produce a mechanical steady state before application of the initial MR radio frequency pulse and motion-sensitizing gradients. The initial phase of the symmetric motion-sensitizing gradients will vary by ±π. The following image acquisition parameters are typical: pulse repetition time (TR) cardia gated ˜1000 ms for a subject with a heart rate of 60 beats/minute ms, echo delay time (TE) 24 ms, slice thickness 5.0 mm, 128-256 phase encoding views with an image acquisition time of about ˜90 s, NEX ˜2-4, gradient field of ˜900-3500 mGauss. Duty cycle of the coil 10 will be ˜5% of the TR time. This is calculated by multiplying the motion-sensitizing period T by the number of cycle and dividing by TR.

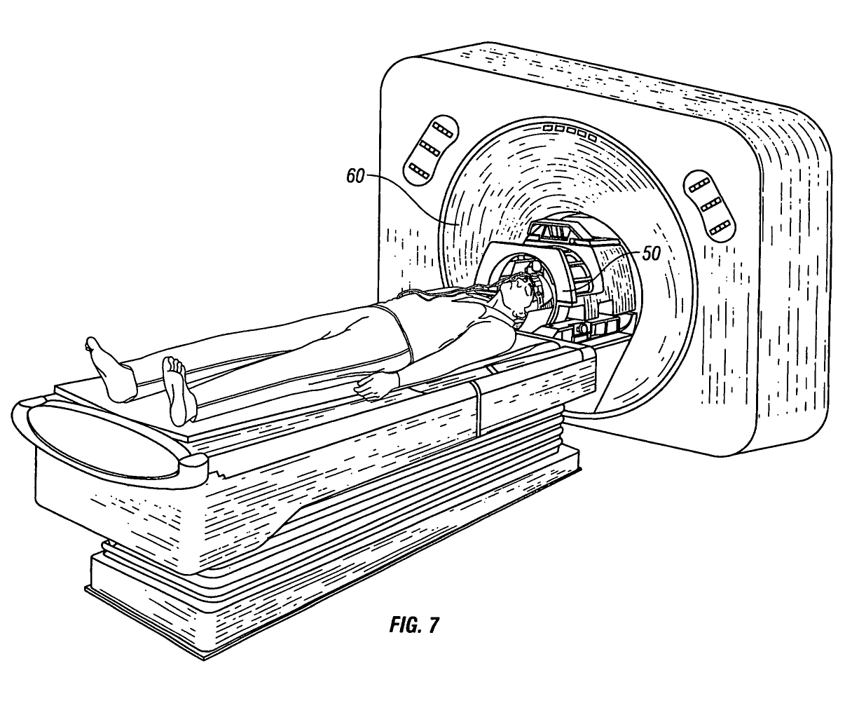

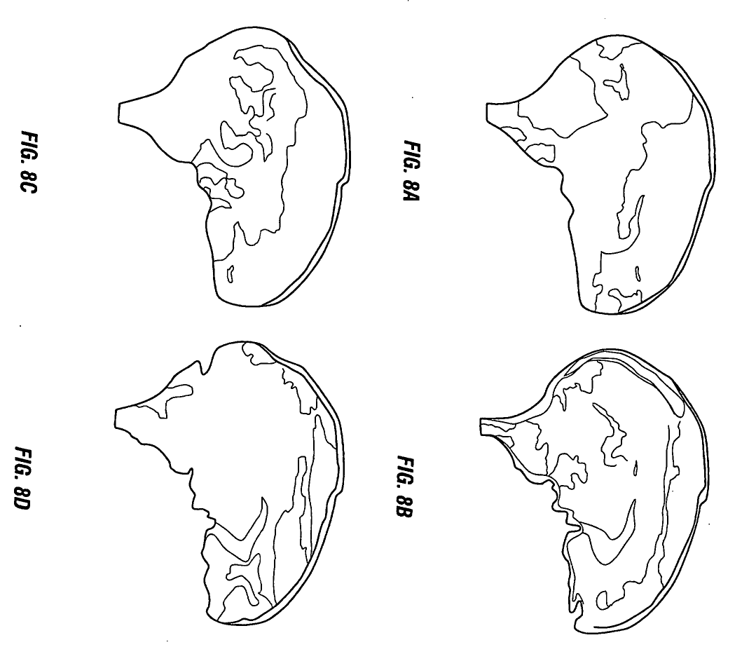

FIG. 7 illustrates patient observation during acoustical excitation of skull 30. This figure illustrates patient with the apparatus of FIG. 1 attached placed within standard MR “birdcage” 50 and passed into MR apparatus 60. FIG. 8 is representative scans brain scans of volunteer individuals taken utilizing the process of this invention.

Referring to FIG. 5, software written in National Instruments LabVIEW is used to output the excitation waveform that drives the excitation coil 10. The software creates the waveform and controls a digital-to-analog converter 72 (National Instruments PCI-6070E) that outputs the waveform. A current stabilized amplifier 74 is used to generate the currents necessary to drive the excitation coil. A highpass filter 73 is used to prevent DC currents from driving the coil (see FIG. 5).

The software allows the user to excite the coil before the actual MRI acquisition begins. The waveform was designed to minimize currents during the period of the MRI sequence where the RF pulse is output and during data acquisition. It was found that any currents present during these parts of the sequence produced artifacts in the MRI images.

Data Analysis

Referring to FIG. 5, a block diagram of the data analysis here present is illustrated. MR P files 90 are processed by fast Fourier transform (inversion) software. Phase unwrapping then occurs to eliminate ambiguities of phase signal redundancy. As will be set forth below, three discrete types of processing are possible utilizing Hilbert transforms 95, local wave length 96 and shear modulus 97. What follows is a theoretical explanation of these computer confined techniques.

A magnetic-field gradient results in a phase shift φ of the NMR signal and is given by

$$ϕ = γ {\int }_{0}^{τ} {G}_{τ} ( t ) \cdot r ( t ) ⅆ t$$

where γ is the gyromagnetic ratio for a proton, Gτ is the magnetic gradient field, τ is the time duration of the gradients while r(t) describes the position of the nuclear spins as a function of time. The following analysis assumes that the tissue properties are locally isotropic with the stress-strain relationship defined by a Hookean relationship. A stationary vibration field will be allowed to evolve prior to sampling the magnetization field by the receiver coil. Considering the case where the acoustic source is periodic and applied for a sufficient period to damp transients then the deviation from equilibrium of an isochromat is determined by local wave properties. These are modified from the source by attenuation, reflection and scattering resulting in a mixture of transverse and longitudinal waves determining the local dyadic strain tensor values

The local stress-strain relation is given by the following tensor equation 30
F=λ(∇ε)1+2 μE

since the time varying body force F1 is

$${F}_{1} = ∇ \cdot F = {ρ}_{0} \frac{{\partial }^{2} ɛ}{\partial {t}^{2}}$$

which becomes

$$( λ + μ ) ∇ ∇ \cdot ɛ + μ ∇ \cdot ∇ ɛ = {ρ}_{0} \frac{{\partial }^{2} ɛ}{\partial {t}^{2}}$$

by considering the irrotational field the above equation becomes

$$∇ \cdot ∇ ɛ = \frac{1}{{c}_{l}^{2}} \frac{{\partial }^{2} ɛ}{\partial {t}^{2}}$$

and by consideration of the solenoidal field the wave equation becomes

$$∇ \cdot ∇ ɛ = \frac{1}{{c}_{l}^{2}} \frac{{\partial }^{2} ɛ}{\partial {t}^{2}}$$

$$where$$

$$\frac{{c}_{l}}{{c}_{t}} = {[ \frac{2 ( 1 - σ )}{1 - 2 σ} ]}^{\frac{1}{2}}$$

$$and$$

$${c}_{t}^{2} = \frac{Y}{2 ( 1 + σ ) {ρ}_{0}}$$

$$and$$

$${c}_{l}^{2} = \frac{B + \frac{4}{3} μ}{{ρ}_{0}} = \frac{Y ( 1 - σ )}{( 1 + σ ) ( 1 - 2 σ ) {ρ}_{0}}$$

hence it possible to isolate for each voxel the Young's modulus and the Poisson ratio.

The spin density ρ(r) is a vector field and related to the NMR signal in the receiver coil. The variation in the spin density is proportional to the strain tensor in a manner

$$ρ ( r ) ∝ ( \begin{matrix}{ɛ}_{xx} & 0 & 0 \\ 0 & {ɛ}_{yy} & 0 \\ 0 & 0 & {ɛ}_{zz}\end{matrix} )$$

following the assumption of local isotropy.

By subtracting the spin density ρ with the applied motion sensitizing gradient acoustic strain field from the spin density with the motion sensitizing gradient opposite in phase, the phase representation of the strain field can be derived. The above analysis makes the assumption of tissue isotropy. This can be further extended by taking the trace of the strain tensor resulting in a mean local strain εav.

$$Tr ( \begin{matrix}{ɛ}_{xx} & 0 & 0 \\ 0 & {ɛ}_{yy} & 0 \\ 0 & 0 & {ɛ}_{zz}\end{matrix} ) = {ɛ}_{av}$$

This can be further simplified to give the average strain for the whole brain. Discrimination of white and gray matter (WM, GM) might be possible using a histogram fitting technique so that average WM and GM properties could be defined. The magnitude of MR gradient is set to be synchronized with the acoustic strain field induced by the HeadWave coil and may described by
| G̅ₜ(t)|=±|G|;tε{nT,(2n+)T/2, or (2n+1)T/2,(n+1)T}

where n=0, 1, 2 . . . N−1, and T=2π/ω. The phase shift in the received signal from the stationary displacement field induced in the tissue by the coil vibration is given by

$$ϕ ( r , α ) = \frac{2 γ NT ( G \cdot ξ )}{π} sin ( k \cdot r + α )$$

where k is the wavenumber, r is the distance from the source, α is the phase lag between the MR gradient and the vibration coil and ζ is the local tissue displacement. A displacement field ζ, can be obtained after inverse Fourier transformation of the k-space complex image and subtraction of the two out of phase motion sensitized gradients images. The reconstructed phase subtraction image allows derivation of the wavelength λ and wavenumber (k) (λ=2π/|k|) by taking the Hilbert transformation (See FIG. 5 at 95) and calculating the instantaneous phase followed calculation of the shear modulus G. The Young's modulus may be calculated from the shear modulus and by assumption of tissue isotropy (See FIG. 5 at 97). By taking images in the two other orthogonal directions, the tissue properties can be isotropically determined. Further images taken in the cross directions allows anisotropic determination of tissue properties.

The equation describing the displacement in an isotropic medium is the Navier equation as follows

$$∇ \cdot μ ∇ u + ∇ ( λ + μ ) ∇ \cdot u = ρ \frac{{\partial }^{2} u}{\partial {t}^{2}}$$

where λ and λ are the Lamé constants. This can be solved directly as a forward problem. The inversion of the 3D isotropic wave equation solving for λ and μ, can be solved using a least squares minimization method 15, 17.

A tensor, Eeff of Young's moduli (E) may be derived with MR by altering the gradient combination (X, Y, Z, X+Y, X+Z, Y+Z). Eeff is a symmetric second order tensor with the dominant directions and magnitudes determinable from the tensor eigenvalues and eigenvectors on a pixel-by-pixel basis. The solution of the full 3D wave equation for the velocity vector field followed by calculation of the full Young's tensor Eeff adds a further order of complexity not only in the data analysis but also in the data acquisition where the complete tensor strain field must be obtained.

Outcome Measures

Primary Outcome Measure

The phase difference images (O) represent the primary outcome measure. Increasing levels of complexity will be developed from single slice single plane data to 3D volume acquisition in three orthogonal directions. The most complex level of data acquisition possible consists of tensor acquisition in six independent directions in each plane.

Secondary Outcome Measures

The following data will be derived from the phase difference images

[1] Wavelength (λ) of the acoustic wave in different areas of the tissue together with derivation of the shear modulus (μ) where μ=(νλ)²ρ (ν is the excitation frequency, ρ is the medium density)

[2] Displacement Field (ξ)

[3] Derived material properties in terms of the Lame coefficients for an isotropic medium

Statistical Analysis

Determination of Sample Size (Power Analysis)

We are determining some indication of the random error involved in MRE following simulation experiments using a 2% agarose phantom skull 30 when multiple measurements are made. This random error most likely will be an underestimate of the random error obtained across a patient population since the variance from measurement in patients will be added to the variance inherent in MRE measurement. Further the biological variance is likely to be the dominant component.

We are undertaking a healthy volunteer investigation to acquire a normal population data. At the present time, the total number of subjects required is unclear until data analysis gives some indication of the data variance involved in MRE. By the central limit theorem, analysis of population sizes of −30 or greater should approach a Gaussian distribution allowing the application of normal statistics to MRE data analysis. This will be of importance in allowing standard brain imaging analysis techniques such as SPM (statistical parametric mapping). SPM fully takes into account multiple comparisons procedures by the application of Gaussian fields. The normal volunteer data acquired will allow appropriate power calculations to be generated in the evaluation of subsequent clinical hypotheses.

Methods Used to Analyze Outcomes

The following statistical considerations will be applied to analysis of the primary and secondary outcome variables

[1] Each subject will be treated as independent so that single global subject dependent variables will be compared by normal univariate statistics.

[2] All multiple variables from single subjects will have appropriate multiple comparison techniques applied. For imaging data, this will entail that each of the independent scalar component of either, the derived vector or tensor fields from the primary or secondary outcome variables will be analyzed using Gaussian field theory and statistical parametric methods. Statistical parametric methods take into account necessary multiple comparison corrections and the data will be analyzed with SPM, a standard statistical parametric package.

Validation of MRE in Healthy Volunteers

Following the above data analysis the values derived from MRE can be compared with the experimentally determined Lame coefficients and Young's moduli for central nervous tissue available in the literature (See Hagermann A, Rohr, K, Stiehl, H S, Spetzger, U, Gilsbach, J M. Biomechanical modeling of the human head for physically-based, non rigid image registration. IEEE TRANS MED IMAGING 1999; 18:875-884.).

FIG. 8A through 8D are graphic depictions of a MRE single slice technique on a healthy volunteer showing ipsilateral sagittal acquisition. The MR imaging was performed using a cardiac gated, phase contrast, gradient echo sequence 1.5T, TE 26 ms, FOV 18 cm, 256×128, slice thickness 5.0 mm with motion-encoding gradients (7 to 2 cycles, 3.5 G/cm) applied during the TE period. All phase images are windowed with land marking slice acquisition through the point of actuator apposition in the axial plane. In sagittal slice acquisition the motion encoding gradients are in the frequency direction.

FIG. 8A illustrates an excitation frequency 125 Hz, Gradient 3.5 G/cm, windowed to ±1.4 radians and phase unwrapped showing areas of high and low phase accumulation secondary to the transmitted transverse acoustic wave, pre-excitation 117 msec. This is a schematic of the phase measurement in a sagittal brain slice after phase unwrapping and windowing to +/−1.4 radians.

FIG. 8B illustrates a Hilbert Transform of FIG. 8A showing the 90° phase shift. This allows the mathematical generation of the 90 degree quadrature image.

FIG. 8C illustrates phase unwrapping of the Hilbert Transform of FIG. 8B. After further phase unwrapping the Hilbert transform and combining FIGS. 8A and 8B, the instantaneous phase can be derived on a pixel-by-pixel basis. By differentiating the local phase we get the local frequency or wavenumber from which the shear modulus can be calculated as in FIG. 8D.

FIG. 8D illustrates the shear modulus map derived from the local spatial frequency or wavenumber map calculated by differentiating the instantaneous phase and applying μ=ρ(λf)².

What is claimed is:

1. A method for magnetic resonance elastography of at least a section of the brain comprising the steps of:
examining the head of a patient in vivo in a magnetic resonance device;
vibrating the head of the patient during the examination at a selected frequency between 125 hertz and 500 hertz;
observing and plotting phase alteration of voxel isochromats at the selected frequency to obtain phase patterns; and,
measuring the phase patterns across at least the section of the brain.

2. The method for magnetic resonance elastography of at least a section of the brain according to claim 1 wherein the measuring by observing phase patterns includes:
repeating the examining, vibrating, observing and plotting, and measuring steps for a group of individuals; and,
comparing the measuring of the phase patterns from one individual to other individuals.

3. The method for magnetic resonance elastography of at least a section of the brain according to claim 1 wherein the measuring by observing phase patterns includes:
analyzing the phase patterns utilizing Hilbert transforms.

4. The method for magnetic resonance elastography of at least a section of the brain according to claim 1 wherein the measuring by observing phase patterns includes:
analyzing the phase patterns by utilizing the shear modulus.

5. The method for magnetic resonance elastography of at least a section of the brain according to claim 1 wherein the measuring by observing phase patterns includes:
analyzing the phase patterns by utilizing the local wavelength.

6. The method for magnetic resonance elastography of at least a section of the brain according to claim 1 wherein:
the observing and plotting phase alteration of voxel isochromats occurs after vibrating the head of the patient for about a time period of 5-200 msec.

7. A method for magnetic resonance elastography of at least a section of the brain comprising the steps of:
affixing a coil to the head of the patient in a magnetic resonance device having a magnetic field;
passing alternating current through the coil to cause vibrational energy to pass from the coil to the head of the patient at a selected frequency between 125 hertz and 500 hertz;
after the passing step, examining the head of a patient in the magnetic resonance device;
observing and plotting phase alteration of voxel isochromats at the selected frequency to obtain phase patterns; and,
measuring the elasticity of the brain by observing the phase patterns across at least the section of the brain.

8. The method for magnetic resonance elastography according to claim 7 and wherein:
observing and plotting phase alteration of voxel isochromats at the selected frequency to obtain phase patterns immediately after passing of the alternating current through the coil has ceased but before vibrational energy within the head of the patient dissipates.

9. The method for magnetic resonance elastography according to claim 7 and wherein the affixing of a coil to the head of the patient includes:
placing a shaft through the coil to receive vibrations from the coil;
placing a probe in rigid contact with a shaft at one portion and preloading the probe into contact with a human skull at another portion; and,
vibrating the coil to impart vibrations through the shaft to the probe to vibrate in vivo a human brain within the skull.

10. The method for magnetic resonance elastography according to claim 9 and wherein the preloading of the probe into contact with the human skull includes:
preloading the probe into contact with the acoustic window of the human skull.

11. A method for magnetic resonance elastography of at least a section of the brain comprising the steps of:
examining the head of a patient in vivo in a magnetic resonance device;
observing the periodicity of the patient's heartbeat for determining a sampling interval with respect to the patient's heartbeat;
vibrating the head of the patient immediately before a sampling interval at a selected frequency between 125 hertz and 500 hertz;
observing and plotting phase alternation of voxel isochromats at the selected frequency to obtain phase patterns; and,
measuring by observing the phase alternation across at least the section of the brain.

12. The method for magnetic resonance elastography of at least a section of the brain according to claim 11 comprising the further steps of:
ceasing the vibrating immediately before the observing and plotting step.

13. An apparatus for improved magnetic resonance analysis of the brain during magnetic resonance examination comprising:
a mounting for preloading a probe on to the cranium of the patient in a magnetic resonance device;
a coil affixed to the probe for passing vibrations from the coil to the probe; and,
means for passing an alternating current through the coil in the range of 125 hertz to 500 hertz to cause the coil to vibrate within the magnetic field of the magnetic resonance device and pass the vibrations of the coil to the probe.

14. The apparatus for improved magnetic resonance analysis of the brain during magnetic resonance examination according to claim 13 and wherein means for passing alternating current through the coil includes:
a high pass filter and a current stabilized amplifier.
